(19) United States
(12) Patent Application Publication
(10) Pub. No.: US 2019/0298506 A1
(43) Pub. Date: Oct. 3, 2019

(54) Urinary Meatus Shielding Device

(71) Applicant: Karen M. Catena, Scotch Plains, NJ (US)

(72) Inventor: Karen M. Catena, Scotch Plains, NJ (US)

(21) Appl. No.: 15/937,563

(22) Filed: Mar. 27, 2018

(51) Int. Cl. A61F 2/00

(52) U.S. Cl. CPC A61F 2/0009; A61F 2002/0081; A61F 2220/005; A61F 2230/0034; A61F 2250/0078

(57) ABSTRACT

A urinary meatus shielding device for preventing urinary tract infections includes a panel that is finely meshed so that the panel is configured to prevent passage of a microorganism through the panel. A first coupler is coupled to a first side of the panel so that the panel is configured to couple to a pelvic region of a female user. The panel is positioned over an external urethral orifice of the user to prevent the microorganism from entering a urethra of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art including information disclosed under 37 CFR 1.97 and 1.98

The disclosure and prior art relates to shielding devices and more particularly pertains to a new shielding device for preventing urinary tract infections.

### BRIEF SUMMARY OF THE INVENTION

An embodiment of the disclosure meets the needs presented above by generally comprising a panel that is finely meshed so that the panel is configured to prevent passage of a microorganism through the panel. A first coupler is coupled to a first side of the panel so that the panel is configured to couple to a pelvic region of a female user. The panel is positioned over an external urethral orifice of the user to prevent the microorganism from entering a urethra of the user.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a urinary meatus shielding device according to an embodiment of the disclosure.

FIG. 2 is a bottom view of an embodiment of the disclosure.

FIG. 3 is a top view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

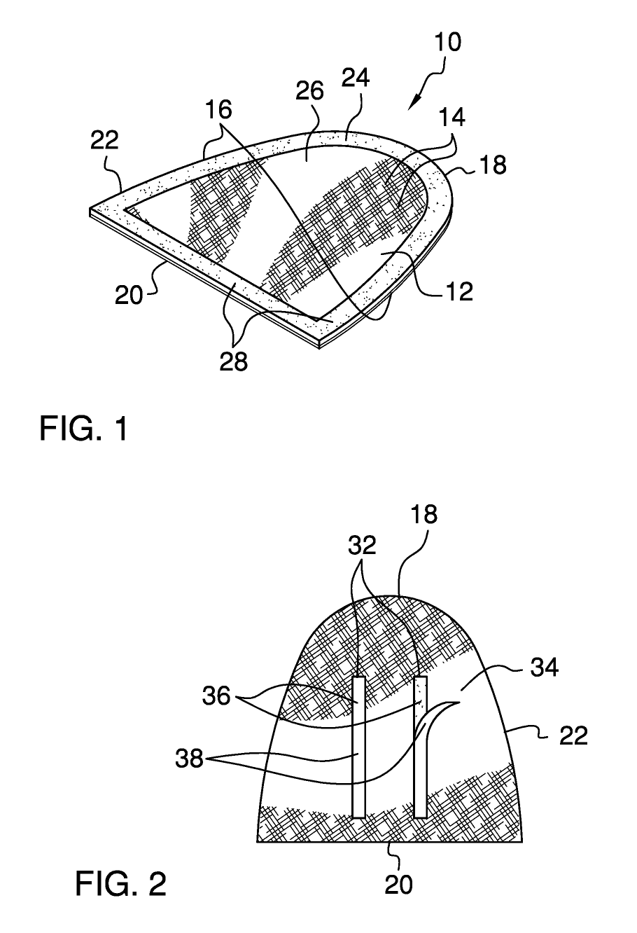

With reference now to the drawings, and in particular to FIGS. 1 through 5 thereof, a new shielding device embodying the principles and concepts of an embodiment of the disclosure and generally designated by the reference numeral 10 will be described.

As best illustrated in FIGS. 1 through 5, the urinary meatus shielding device 10 generally comprises a panel 12 that is finely meshed. The panel 12 is configured to prevent passage of a microorganism through the panel 12. The panel 12 has pores 14 that are sized to prevent passage of the microorganism through the panel 12 and to allow passage of urine through the panel 12. The panel 12 has opposing sides 16 that extend between a top 18 and a bottom 20. The opposing sides 16, the top 18, and the bottom 20 define a perimeter 22 of the panel 12. The bottom 20 is linear while the opposing sides 16 and the top 18 are arcuate so that the panel 12 is half-oval shaped.

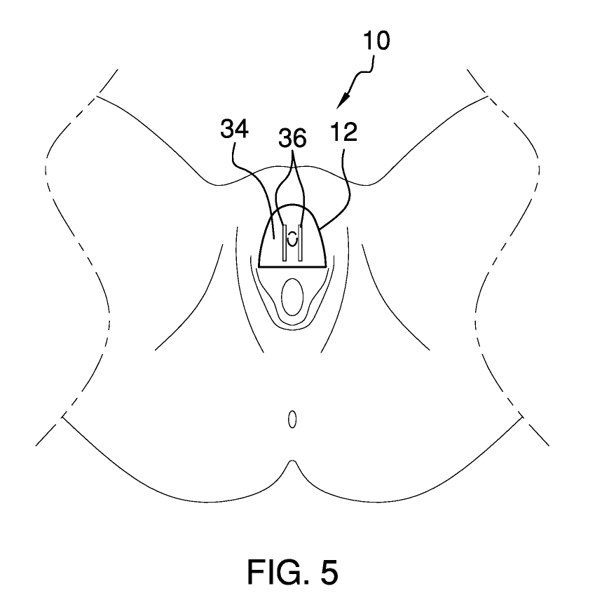

A first coupler 24 is coupled to a first side 26 of the panel 12 so that the panel 12 is configured to couple to a pelvic region of a female user. The panel 12 is positioned over an external urethral orifice of the user, as shown in FIG. 5. The panel 12 prevents the microorganism, such as a microorganism present in the vaginal region or anal region of the user, from entering a urethra of the user. Preventing entry of the microorganism into the urethra prevents a urinary tract infection. The pores 14 in the panel 12 allow passage of the urine through the panel 12 so that the user can urinate without removing the panel 12.

The first coupler 24 comprises a first strip 28 that is coupled to the panel 12. The first strip 28 extends along the opposing sides 16 and the top 18 of the panel 12, as shown in FIG. 2. The first strip 28 comprises adhesive. The first strip 28 is configured to adhesively couple the panel 12 to the pelvic region of the user so that the bottom 20 of the panel 12 is positioned between the external urethral orifice and a vaginal opening of the user. The panel 12 is positioned over the external urethral orifice to prevent the microorganism from entering the urethra. In one embodiment, as shown in FIG. 1, the first strip 28 extends around the perimeter 22 of the panel 12.

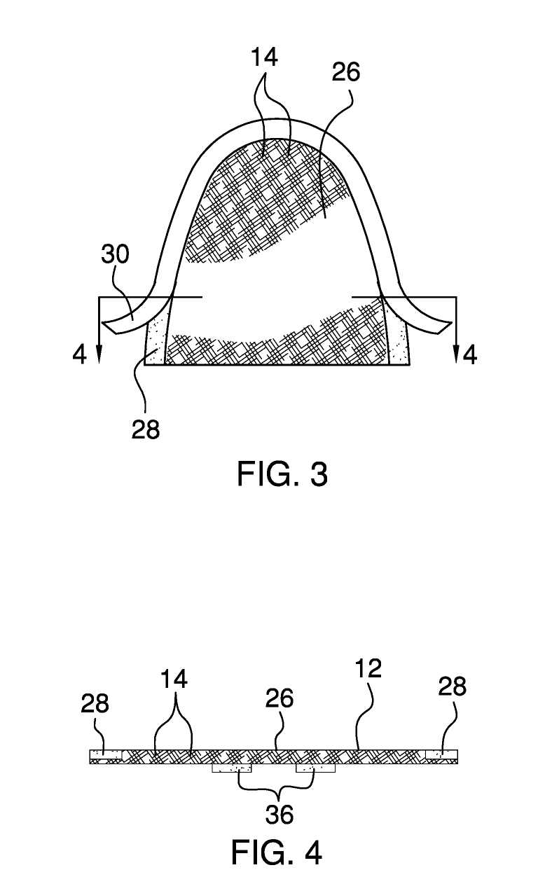

A second strip 30 is reversibly coupled to the first strip 28, as shown in FIG. 3.

The second strip 30 is positioned to be selectively removed to position the first strip 28 to couple to the pelvic region of the user.

A second coupler 32 is coupled to a second side 34 of the panel 12, as shown in FIG. 2, so that the panel 12 is configured to selective couple to an undergarment. The panel 12 is positioned over the external urethral orifice of the user when the user is wearing the undergarment, positioning the panel 12 to prevent the microorganism from entering the urethra and providing an alternate method of using the panel 12.

The second coupler 32 comprises a pair of third strips 36 that are coupled to the panel 12. The third strips 36 comprise adhesive. Each third strip 36 extends perpendicularly from proximate to the bottom 20 toward the top 18 of the panel 12. The third strips 36 are positioned to bracket the external urethral orifice when the user is wearing the undergarment.

Each of a pair of fourth strips 38 is reversibly coupled to a respective third strip 36. Each fourth strip 38 is positioned to be selectively removed to position the respective third strip 36 to couple to the undergarment.

In use, the first strip 28 is configured to adhesively couple the panel 12 to the pelvic region of the user. The bottom 20 of the panel 12 is positioned between the external urethral orifice and the vaginal opening of the user. The panel 12 is positioned over the external urethral orifice to prevent the microorganism from entering the urethra, thus preventing the urinary tract infection.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A urinary meatus shielding device comprising:
a panel, said panel being finely meshed wherein said panel is configured for preventing passage of a microorganism through said panel; and
a first coupler coupled to a first side of said panel wherein said panel is configured for coupling to a pelvic region of a female user such that said panel is positioned over an external urethral orifice of the user for preventing the microorganism from entering a urethra of the user.

2. The device of claim 1, further including said panel having pores, said pores being sized for preventing passage of the microorganism through said panel and for allowing passage of urine through said panel.

3. The device of claim 1, further including said panel having opposing sides extending between a top and a bottom defining a perimeter of said panel, said bottom being linear, said opposing sides and said top being arcuate such that said panel is half-oval shaped.

4. The device of claim 3, further including said first coupler comprising a first strip coupled to said panel, said first strip extending along said opposing sides and said top of said panel, said first strip comprising adhesive, wherein said first strip is positioned on said panel such that said first strip is configured for adhesively coupling said panel to the pelvic region of the user such that said bottom of said panel is positioned between the external urethral orifice and a vaginal opening of the user positioning said panel over the external urethral orifice for preventing the microorganism from entering the urethra.

5. The device of claim 4, further including said first strip extending around said perimeter of said panel.

6. The device of claim 4, further including a second strip reversibly coupled to said first strip, wherein said second strip is positioned on said first strip such that said second strip is positioned for being selectively removed positioning said first strip for coupling to the pelvic region of the user.

7. The device of claim 3, further including a second coupler coupled to a second side of said panel such that said panel is configured for selective coupling to an undergarment such that said panel is positioned over the external urethral orifice of the user when the user is wearing the undergarment for preventing the microorganism from entering the urethra.

8. The device of claim 7, further including said second coupler comprising a pair of third strips coupled to said panel, said third strips comprising adhesive, each said third strip extending perpendicularly from proximate to said bottom toward said top of said panel such that said third strips bracket the external urethral orifice when the user is wearing the undergarment.

9. The device of claim 8, further including a pair of fourth strips, each said fourth strip being reversibly coupled to a respective said third strip, wherein said fourth strips are positioned on said third strips such that each said fourth strip is positioned for being selectively removed positioning said respective said third strip for coupling to the undergarment.

10. A urinary meatus shielding device comprising:
a panel, said panel being finely meshed wherein said panel is configured for preventing passage of a microorganism through said panel, said panel having pores, said pores being sized for preventing passage of the microorganism through said panel and for allowing passage of urine through said panel, said panel having opposing sides extending between a top and a bottom defining a perimeter of said panel, said bottom being linear, said opposing sides and said top being arcuate such that said panel is half-oval shaped;
a first coupler coupled to a first side of said panel wherein said panel is configured for coupling to a pelvic region of a female user such that said panel is positioned over an external urethral orifice of the user for preventing the microorganism from entering a urethra of the user, said first coupler comprising a first strip coupled to said panel, said first strip extending along said opposing sides and said top of said panel, said first strip comprising adhesive, wherein said first strip is positioned on said panel such that said first strip is configured for adhesively coupling said panel to the pelvic region of the user such that said bottom of said panel is positioned between the external urethral orifice and a vaginal opening of the user positioning said panel over the external urethral orifice for preventing the microorganism from entering the urethra, said first strip extending around said perimeter of said panel;
a second strip reversibly coupled to said first strip, wherein said second strip is positioned on said first strip such that said second strip is positioned for being selectively removed positioning said first strip for coupling to the pelvic region of the user;
a second coupler coupled to a second side of said panel such that said panel is configured for selective coupling to an undergarment such that said panel is positioned over the external urethral orifice of the user when the user is wearing the undergarment for preventing the microorganism from entering the urethra, said second coupler comprising a pair of third strips coupled to said panel, said third strips comprising adhesive, each said third strip extending perpendicularly from proximate to said bottom toward said top of said panel such that said third strips bracket the external urethral orifice when the user is wearing the undergarment;
a pair of fourth strips, each said fourth strip being reversibly coupled to a respective said third strip, wherein said fourth strips are positioned on said third strips such that each said fourth strip is positioned for being selectively removed positioning said respective said third strip for coupling to the undergarment; and
wherein said first strip is positioned on said panel such that said first strip is configured for adhesively coupling said panel to the pelvic region of the user such that said bottom of said panel is positioned between the external urethral orifice and the vaginal opening of the user positioning said panel over the external urethral orifice for preventing the microorganism from entering the urethra.
